(19) United States
(12) Patent Application Publication
(10) Pub. No.: US 2026/0116860 A1
(43) Pub. Date: Apr. 30, 2026

(54) METHOD FOR PRODUCING ISOXAZOLINECARBOXYLIC ACID DERIVATIVES

(71) Applicant: BAYER AKTIENGESELLSCHAFT, LEVERKUSEN (DE)

(72) Inventors: Anton LISHCHYNSKYI, Leverkusen (DE); Frank MEMMEL, Leverkusen (DE); Mark James FORD, Leverkusen (DE); Klaus Bernhard HAAF, Leverkusen (DE)

(21) Appl. No.: 18/570,761

(22) PCT Filed: Jun. 24, 2022
(86) PCT No.: PCT/EP2022/067334

§ 371 Date: Dec. 15, 2023

(30) Foreign Application Priority Data
Jun. 29, 2021 (EP) 21182458.6

(51) Int. Cl. C07D 261/04

(52) U.S. Cl. CPC C07D 261/04

(57) ABSTRACT

The present invention relates to a novel process for preparing isoxazolinecarboxylic acid derivatives of the formula (I).

The present invention relates to a novel process for preparing isoxazolinecarboxylic acid derivatives of the formula (I).

Isoxazolinecarboxylic acid derivatives of the general formula (I) are important precursors of active agrochemical ingredients (cf. WO 2018/228985).

The prior art describes numerous cyclization methods for preparation of isoxazolinecarboxylic acid derivatives, for example Tetrahedron Letters, 1991, 6367-6370; Eur. J. Org. Chem. 2008, 5446-5460; Bull. Chem. Soc. Jpn. 1993, 2685. Possible transition states for the cycloaddition are discussed. Also disclosed are yields and isomer ratios depending on the reaction conditions.

If the compounds of the present invention are obtained by one of the methods known from the literature, this results in yields and isomer purities that are inadequate for an industrial scale synthesis.

It was thus an object of the invention to provide a process for preparing isoxazolinecarboxylic acid derivatives of the general formula (I), which is suitable for synthesis on an industrial scale and has a high yield and isomer purity, such that laborious purification methods can be dispensed with.

The object was achieved in accordance with the invention by a process for preparing isoxazolinecarboxylic acid derivatives of the general formula (I)

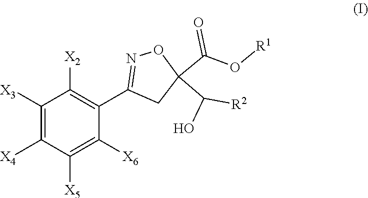

in which
X² is H, C₁-C₄ alkyl, C₁-C₄ fluoroalkyl, C₁-C₄ fluoroalkoxy, C₁-C₄ alkoxy, fluorine, CN,
X³ is H, C₁-C₄ alkyl, C₁-C₄ fluoroalkyl, C₁-C₄ fluoroalkoxy, C₁-C₄ alkoxy, fluorine, chlorine, CN,
X⁴ is H, C₁-C₄ alkyl, C₁-C₄ fluoroalkyl, C₁-C₄ fluoroalkoxy, C₁-C₄ alkoxy, fluorine, CN,
X⁵ is H, C₁-C₄ alkyl, C₁-C₄ fluoroalkyl, C₁-C₄ fluoroalkoxy, C₁-C₄ alkoxy, fluorine, chlorine, CN,
X⁶ is H, C₁-C₄ alkyl, C₁-C₄ fluoroalkyl, C₁-C₄ fluoroalkoxy, C₁-C₄ alkoxy, fluorine, CN,
R¹ is H, C₁-C₄-alkyl,
R² is C₁-C₄-alkyl,
   characterized in that the compounds of the general formula (II)

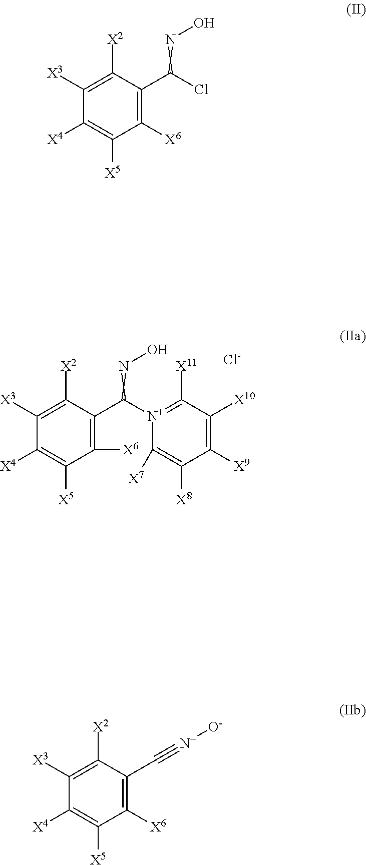

in which
X² to X⁶ have the definitions given above,
X⁷, X⁸, X¹⁰, X¹¹ are independently H or C₁-C₄-alkyl,
X⁹ are H, C₁-C₄-alkyl or N(C₁-C₄-alkyl)₂,
   are reacted with compounds of the formula (III)

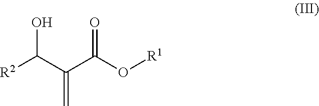

in which
R¹ and R² have the definitions given above,
   with addition of a combination of reagents that enables formation of 2.0 to 4.5 equivalents of a reactive species “R³OMgHal” (IV)-based on compounds of the general formula (II), where
R³ is C₂-C₆-alkyl, unsubstituted or C₁-C₆-alkyl-substituted benzyl and
Hal is halogen,
   to give compounds of the general formula (I).

Preferred definitions of the radicals in the compounds of the general formulae (I), (II) and (III) are as follows:
X² is H, methyl, trifluoromethyl, difluoromethyl, difluoromethoxy, trifluoromethoxy, fluorine, methoxy, CN,
X³ is H, methyl, trifluoromethyl, difluoromethyl, difluoromethoxy, trifluoromethoxy, fluorine, chlorine, methoxy, CN,
X⁴ is H, methyl, trifluoromethyl, difluoromethyl, difluoromethoxy, trifluoromethoxy, fluorine, methoxy, CN,
X⁵ is H, methyl, trifluoromethyl, difluoromethyl, difluoromethoxy, trifluoromethoxy, fluorine, chlorine, methoxy, CN,
X⁶ is H, methyl, trifluoromethyl, difluoromethyl, difluoromethoxy, trifluoromethoxy, fluorine, methoxy, CN,
X⁷, X⁸, X¹⁰, X¹¹ are independently H, methyl, ethyl,
X⁹ is H, methyl, ethyl or N(methyl)₂
R¹ is H, methyl, ethyl, i-propyl, i-butyl,
R² is methyl, ethyl.

Preferably, the compound of the general formula (IV) is generated by one of the following combinations of reagents:
R⁴Mg Hal and R³OH or
MgHal₂ and R³OM or
MgHal₂ and Mg(OR³)₂, where
R³ is C₂-C₆-alkyl, benzyl, methylbenzyl,
Hal is halogen,
M is alkali metal,
R⁴ is C₁-C₆-alkyl, aryl, benzyl, allyl, vinyl.

Alternatively, the compound of the general formula (IV) is generated by the following combination of reagents:
R⁵MgHal and R⁶R⁷CO, where
   R⁵ is C₁-C₆-alkyl, aryl, benzyl,
   R⁶, R⁷ are H, C₁-C₆-alkyl, aryl, and the resulting radical definition
   R³ is R⁵R⁶R⁷C.

Preferred definitions of radicals for the compounds of the general formula (IV), prepared via the alternative combination of reagents:
R⁵MgHal and R⁶R⁷CO,
   are as follows:
R⁵ is C₁-C₄-alkyl, phenyl, benzyl, p-tolyl,
R⁶, R⁷ is H, C₁-C₄-alkyl, phenyl.

Particularly preferred definitions of the radicals in the compounds of the general formulae (I), (II) and (III) are as follows:
X² is H,
X³ is H, methyl, trifluoromethyl, difluoromethyl, fluorine, chlorine, methoxy, CN,
X⁴ is fluorine, H,
X⁵ is H, methyl, trifluoromethyl, difluoromethyl, fluorine, chlorine, methoxy, CN,
X⁶ is H,
X⁷, X⁸, X¹¹ are independently H, methyl, ethyl,
X⁹ is H, N(methyl)₂
X¹⁰ is H,
R¹ is H, methyl, i-propyl, i-butyl,
R² is methyl.

More preferably, the compound of the general formula (IV) is generated by one of the following combinations of reagents:
R⁴MgHal and R³OH or
MgHal₂ and R³OM or
MgHal₂ and Mg(OR³)₂, where
R³ is C₂-C₄-alkyl,
Hal is bromine, chlorine,
M is alkali metal,
R⁴ is C₁-C₄-alkyl, phenyl, benzyl, p-tolyl, vinyl.

Very particularly preferred definitions of the radicals in the compounds of the general formulae (I), (II) and (III) are as follows:
X² is H,
X³ is H, fluorine,
X⁴ is H, fluorine,
X⁵ is H, fluorine,
X⁶ is H,
X⁸ is H, methyl, ethyl,
X⁷, X¹¹ is independently H, methyl,
X⁹, X¹⁰ is H,
R¹ is H, methyl, i-butyl,
R² is methyl.

Even more preferably, the compound of the general formula (IV) is generated by one of the following combinations of reagents:
R⁴MgHal and R³OH or
MgHal₂ and R³OM or
MgHal₂ and Mg(OR³)₂, where
R³ is i-propyl, i-butyl,
Hal is bromine, chlorine,
M is sodium,
R⁴ is methyl, ethyl, n-butyl, i-propyl.

Most preferred definitions of the radicals in the compounds of the general formulae (I), (II) and (III) are as follows:
X² is H,
X³ is fluorine,
X⁴ is H,
X⁵ is fluorine,
X⁶ is H,
X⁷, X⁸, X¹¹ is independently H, methyl,
X⁹, X¹⁰ is H,
R¹ is H, methyl,
R² is methyl.

The prior art discloses some compounds of the formula (I)

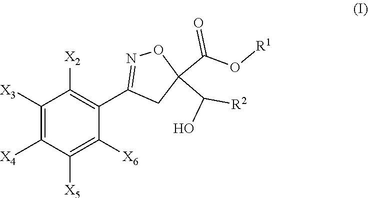

where the radicals correspond to the above-specified general, preferred, more preferred, even more preferred and most preferred definitions,
and are in the form of isomer mixtures:

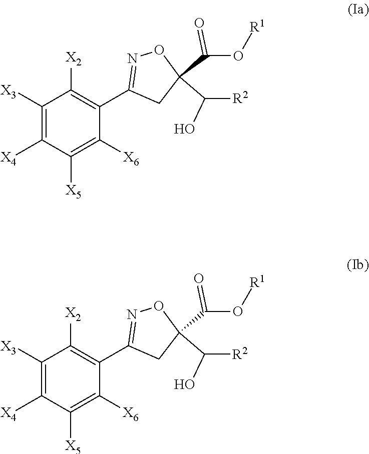

The isomer ratio between (Ia) and (Ib) varies; in general, (Ia) is in excess. This desired excess is increased compared to the prior art by the optimized reaction conditions. For the achievement of a high diastereomeric excess, it is especially advantageous when there are 2.0 to 4.5 equivalents of a reactive species “R³OMgHal” (IV) in the reaction mixture.

The compounds of the formula (I) may be isolated as the corresponding ester or as the carboxylic acid after a hydrolytic workup.

The diastereomeric ratio up to 100:0 may be achieved in the compounds of the formula (I) via an enrichment by crystallization.

The present invention further provides compounds of the formula (Ia) and (Ib) in any mixing ratio except 1:1:

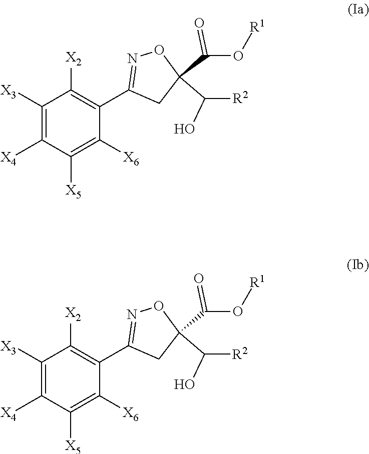

where the radicals correspond to the abovementioned particularly preferred, very particularly preferred and most preferred definitions, excluding methyl 3-(3,5-difluorophenyl)-5-(1-hydroxyethyl)-4H-isoxazole-5-carboxylate and methyl 3-phenyl-5-(1-hydroxyethyl)-4H-isoxazole-5-carboxylate.

The mixing ratio (Ia):(Ib) is preferably at least 90:1, more preferably at least 95:5, even more preferably at least 99:1.

If the reactant used is enantiomerically pure (S)-(III), it is possible to prepare the compounds of the formulae (Iaa) and (Iba).

The present invention further provides compounds of the formula (Iaa) and (Iba) in any mixing ratio except 1:1:

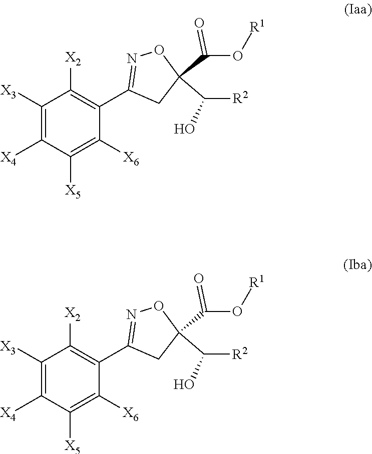

where the radicals correspond to the abovementioned particularly preferred, very particularly preferred and most preferred definitions, excluding methyl 3-(3,5-difluorophenyl)-5-(1-hydroxyethyl)-4H-isoxazole-5-carboxylate and methyl 3-phenyl-5-(1-hydroxyethyl)-4H-isoxazole-5-carboxylate.

The mixing ratio (Iaa):(Iba) is preferably at least 90:1, more preferably at least 95:5, even more preferably at least 99:1.

If the reactant used is enantiomerically pure (R)-(III), it is possible to prepare the compounds of the formulae (Iab) and (Ibb).

The present invention further provides compounds of the formula (Tab) and (Ibb) in any mixing ratio except 1:1:

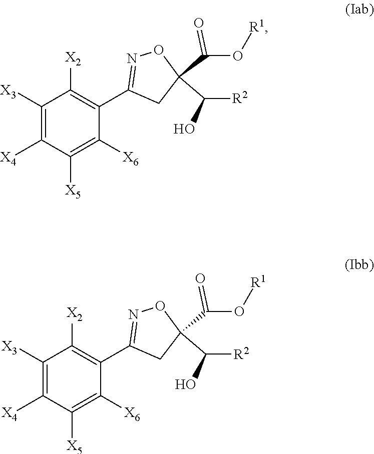

where the radicals correspond to the abovementioned particularly preferred, very particularly preferred and most preferred definitions, excluding methyl 3-(3,5-difluorophenyl)-5-(1-hydroxyethyl)-4H-isoxazole-5-carboxylate and methyl 3-phenyl-5-(1-hydroxyethyl)-4H-isoxazole-5-carboxylate.

The mixing ratio (Iab):(Ibb) is preferably at least 90:1, more preferably at least 95:5, even more preferably at least 99:1.

### Elucidation of the Processes and Intermediate

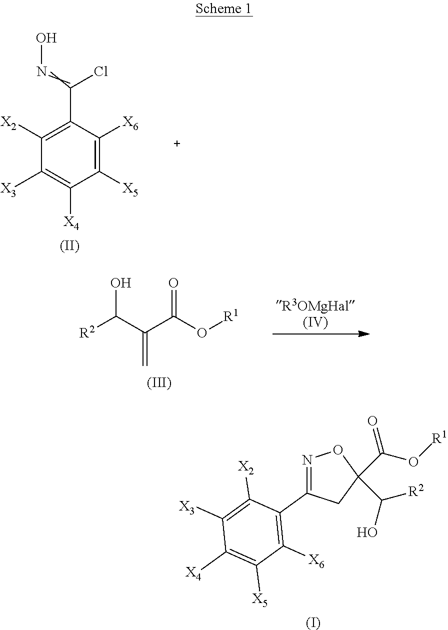

The object was achieved by a process for preparing isoxazolinecarboxylic acid derivatives of the formula (I), characterized in that the compounds of the general formula (II) are reacted with compounds of the formula (III) with addition of a combination of reagents that enables formation of 2.0 to 4.5 equivalents of a reactive species “R³OMgHal” (IV) based on compounds of the general formula (II) to give compounds of the general formula (I) (Scheme 1).

Table 1 shows various possible combinations of reagents, although this selection is not exhaustive.

There are preferably 2.8 to 3.2 equivalents of the reactive species “R³OMgHal” (IV) based on compounds of the general formula (II).

There are more preferably 3 equivalents of the reactive species “R³OMgHal” (IV) based on compounds of the general formula (II).

The cyclization is usually conducted within a temperature range from −25° C. to 70° C., preferably 10° C. to 30° C.

In addition, the cyclization is optionally conducted in the presence of a solvent or diluent or of a solvent mixture. The solvents are preferably toluene, xylene, tetrahydrofuran (THF), isopropyl acetate (i-PrOAc), acetonitrile, methyl tert-butyl ether (MTBE), methyl-THF, ethyl acetate (EtOAc) or mixtures in any ratios thereof.

The compounds of the general formula (III) are prepared via a two-stage process, and are known from the literature. The first stage is a Baylis-Hillman reaction. A relevant literature reference is: Drewes, S. E.; Hoole, R. F. A. [Synthetic Communications, 1985, vol. 15, 12, p. 1067-1074]. For the second stage, a relevant literature reference is: Nascimento et al. (2003, Tetrahedron Asymmetry 14, 311-311).

The compounds of the general formulae (II) and (III) are also known from WO 2018/228985. The preparation of the compounds of the formula (IIa) from (II) is known from Binenfeld, Zlatko; et al Glasnik Hemijskog Drustva Beograd (1966), 31(4-6), 243-50 and Daroszewski, J.; et al Pharmazie (1986), 41(10), 699-702.

### EXAMPLES

The present invention is elucidated in detail by the examples that follow, without restricting the invention thereto.

### Measurement Methods

The products were characterized by ¹H NMR spectroscopy and/or LC-MS (Liquid Chromatography Mass Spectrometry).

The NMR spectra were determined using a Bruker Avance 400 fitted with a flow probe head (volume 60 μl). In individual cases, the NMR spectra were measured with a Bruker Avance II 600.

### Example 1

2-Propanol (48 ml) and THF (61 ml) are initially charged in a 11 reaction vessel at 24° C. under an argon atmosphere. The mixture is cooled down to 0° C. Then 204 ml of a methylmagnesium chloride solution (3M in THF) is added over the course of two hours, while keeping the reaction temperature at 10 to 25° C. (cooling by ice bath). Methane escapes, and a white solid precipitates out. The resultant suspension is stirred further and warmed to 20° C. after the evolution of gas has ended. Then 27.46 g of methyl 3-hydroxy-2-methylenebutanoate is added to the reaction mixture at 20° C. over the course of 30 minutes. In the course of this, the reaction temperature is kept at 18 to 22° C. with a water bath. After the addition, the reaction mixture is stirred further for another 10 minutes, before 240 g of 3,5-difluoro-N-hydroxybenzenecarboximidoyl chloride in a solvent mixture of toluene/THF/i-PrOAc/DBF (16.3% by weight) is added at 20° C., in the course of which the reaction vessel is cooled with an ice bath. On completion of addition, the reaction mixture is worked up by adding the reaction mixture to 40.25 g of HCl (37% by weight) in 83.85 g of water at room temperature. The organic phase is separated off. After rinsing with toluene (25 ml), the mixture is distilled (top temperature 48-58° C., 580 to 160 mbar).

Hydrolysis is effected with 69.4 g of NaOH (20% by weight, 1.7 equivalents). The solution is stirred at 60° C. for 1 hour until it has ended.

For workup, 100 ml of toluene, 100 ml of water and 3.00 g of H₂SO₄ (20% by weight) are added to the reaction mixture, and two phases are formed. The organic phase is separated off, and the water phase (pH˜7) is washed with 50 ml of toluene in order to extract residues of DBF. 200 ml of i-PrOAc is added to the resulting water phase containing the sodium salt of the product, and then 65.09 g of H₂SO₄ (20% by weight) at room temperature and with vigorous stirring, which leads to a biphasic mixture having a pH of 1 of the water phase. The organic phase is separated off, and the water phase is extracted with 50 ml of i-PrOAc in order to remove the remainder of the product. The i-PrOAc phases are combined and distilled under reduced pressure (210 to 200 mbar) at 50° C. while stirring, which led to a suspension that was thick but still efficiently stirrable (after distillation of about 150 ml of i-PrOAc). Then 100 ml of toluene is added and the distillation is continued under reduced pressure (200 to 150 mbar) at 50° C. (about 100 ml of i-PrOAc/toluene mixture was distilled off). The mixture is cooled down to room temperature and left to stand overnight to complete the crystallization. The suspension is then filtered and washed on the filter with a further 50 ml of toluene, which led to a solid material that was dried in a vacuum drying cabinet (40° C., 20 mbar), which led to 45.54 g (98.0%, 80.6% yield) of the product. The (5S)-3-(3,5-difluorophenyl)-5-[(1S)-1-hydroxyethyl]-4,5-dihydro-1,2-oxazole-5-carboxylic acid product was analysed by HPLC and shows a diastereomeric ratio of 97.6:2.4. The mother liquor showed a diastereomeric ratio of (31.3:68.7).

¹H-NMR (400 MHz, DMSO-d₆): δ (ppm)=1.12 (d, J=6.5 Hz, 3H), 3.62 (d, J=17.8, 1H), 3.68 (d, J=17.8, 1H), 4.10 (q, J=6.4 Hz, 1H), 5.30 (bs, 1H), 7.33-7.48 (m, 3H), 13.24 (bs, 1H).

¹⁹F-NMR (376 MHz, DMSO-d₆): δ (ppm)=−108.7 (m, 2F).

### Example 2

A 21 jacketed vessel is initially charged with 74.74 g of magnesium (3.075 equivalents) in THE (11) under a nitrogen atmosphere and at jacket temperature 20° C. Subsequently, 2.5 ml of 2-propylmagnesium chloride solution (2M in THF, 0.005 equivalent) is added and the mixture is stirred for 10 min, then 248.00 g of 2-chloropropane (99%, 3.126 equivalents) is added dropwise. The internal temperature rises to 35° C., and the jacket temperature is adjusted to 10° C. The entirety of the 2-chloropropane is added dropwise within 2 hours. The internal temperature is kept at about 30 to 34° C. by cooling. The magnesium is depleted gradually. After the end of the addition, the temperature drops gradually, and the jacket temperature is increased gradually to 30° C. Stirring of the mixture is continued for a total of two hours at internal temperature 29 to 33° C. until only a few magnesium flakes are present. 185.17 g of 2-propanol (3.075 equivalents) is added dropwise at jacket temperature 15° C. The internal temperature is kept at 30 to 35° C. Constant evolution of gas (propane) is observed. A solid gradually precipitates out. A thick, still-stirrable suspension is formed. After the addition over the course of 1 hour and 30 min, there is a thick grey suspension. The mixture is cooled to 20° C. and 130.40 g of methyl (3S)-3-hydroxy-2-methylenebutanoate (1 equivalent) is added. The mixture is stirred for a further 10 min, and the jacket temperature is adjusted to 10° C. Then a solution of 3,5-difluoro-N-hydroxybenzenecarboximidoyl chloride (15.2%, 1260.30 g) is added dropwise at internal temperature 18 to 20° C. and jacket temperature 10° C. over the course of 1.5 hours. After the end of the addition, the mixture is stirred at 20° C. for 1 hour.

For the workup, the reaction mixture is added to 403 g of ice and 200 g of HCl solution (37% by weight). The organic phase is separated off and concentrated at bath temperature 50° C. and 350 mbar. 314.40 g of sodium hydroxide solution (20% by weight, 1.57 equivalents) is added to the resultant residue, and the mixture is stirred at 60° C. for two hours until the hydrolysis has ended. The solvent is distilled off at 325 mbar and bath temperature 60° C. 450 g of water and 372 g of toluene are added to the residue. After the extraction, the toluene phase is separated off. After acidification with sulfuric acid (500 g, 20% by weight), the water phase is then extracted with i-propyl acetate. The solvent of the organic phase is removed (internal temperature 40 to 61° C., 200 mbar). The resulting suspension is filtered at 20° C. and washed on the filter with a further 280 ml of toluene, which led to a solid material that was dried under air.

The resulting product is (5S)-3-(3,5-difluorophenyl)-5-[(1S)-1-hydroxyethyl]-4,5-dihydro-1,2-oxazole-5-carboxylic acid: 208.5 g (98.1% pure, diastereomeric ratio >99.9:0.1, 75.4% yield).

¹H-NMR (401 MHz, DMSO-d₆): δ (ppm)=1.11 (d, J=6.5 Hz, 3H), 3.61 (d, J=17.8, 1H), 3.67 (d, J=17.8, 1H), 4.10 (q, J=6.4 Hz, 1H), 5.20 (bs, 1H), 7.34-7.45 (m, 3H), 13.28 (bs, 1H).

¹⁹F-NMR (376 MHz, DMSO-d₆): δ (ppm)=−108.7 (m, 2F).

### Example 10

6.79 g (51.2 mmol) of methyl (3S)-3-hydroxy-2-methylenebutanoate was dissolved in 650 ml of dichloromethane under an argon atmosphere at room temperature, and isopropanol was added. The clear solution was cooled to 0° C. Subsequently, 156 ml (156 mmol) of EtMgBr (1M) in THE was slowly added dropwise (exothermic). The solution turned cloudy. Then a solution of 10.00 g (52.2 mmol) of 3,5-difluoro-N-hydroxybenzimidoyl chloride in 100 ml of DCM was slowly added dropwise while stirring (15 min), and the mixture was warmed gradually to room temperature. The mixture turned a distinct yellow colour. TLC in EA/n-heptane 1:1 showed complete conversion after 30 min.

### Workup:

The solution was added to 11 of a 1:1 mixture of 2N HCl and saturated NaCl solution, and extracted twice with 400 ml each time of methylene chloride, dried over Na₂SO₄, filtered and concentrated. The LCMS analysis of the crude product showed a diastereomeric ratio of the products of 86% to 14%.

Chromatography on Silica Gel (n-Hep/EA)

Fr. 1 m=600 mg (4.0%) of lipophilic diastereomer

¹H NMR (CDCl₃): 1.10 (d, 3H, CHCH₃), 2.3 (s br., 1H, OH), 3.53 (d, 1H, CHH isoxazoline), 3.72 (d, 1H, CHH isoxazoline), 3.84b(s, 3H, OCH₃), 4.34 (q, 1H, CHCH₃), 6.88 (tt, 1H, arom. H), 7.22 (m, 2H, arom. H).

Fr. 2 m=7700 mg (51.7%) polar diastereomer

¹H NMR (CDCl₃): 1.29 (d, 3H, CHCH₃), 2.10 (d, 1H, OH), 3.57 (d, 1H, CHH isoxazoline), 3.69 (d, 1H, CHH isoxazoline), 3.84b(s, 3H, OCH₃), 4.24 (q, 1H, CHCH₃), 6.88 (tt, 1H, arom.H), 7.18 (m, 2H, arom. H).

TABLE 1

| | R1, | | | | |
|---|---|---|---|---|---|
| | (III) | | | | |
| | Stereo- | Reagent | | | |
| | chemistry, | combination | | | Diastereomeric |
| Inventive | Equiv. | (IV) | Solvent, temperature | Yield | ratio, dr |
| Example 1 | Methyl, | 3.0 eq | 1. THF, 10-25° C. | 81% | 98:2 |
| | rac, 1 | MeMgCl | 2. THF/toluene/i- | (isolated) | (isolated) |
| | eq | (22% in THF) | PrOAc/DBF, 20° C. | | 93:7 |
| | | 3.05 eq i-PrOH | | | (reaction |
| | | | | | mixture) |
| Example 2 | Methyl, | 3.075 eq Mg, | 1. THF, 30-35° C.; | 75% | 100% |
| | S, 1 eq | 3.075 eq i- | 2. THF/toluene/ | (isolated) | (isolated) |
| | | PrOH, | DBF, 18-20° C. | | 93:7 |
| | | 0.5 mol % i- | | | (reaction |
| | | PrMgCl, 3.126 | | | mixture) |
| | | eq i-PrCl | | | |
| Example 3 | Methyl, | 3.6 eq MgCl₂ | 2. THF, 20-50° C. | 85% | 92:8 |
| | S, 1 eq | 3.5 eq i-PrONa | 3. THF/toluene/i- | (HPLC) | |
| | | (25% in THF) | PrOAc/DBF, 20° C. | | |
| Example 4 | Methyl, | 1.5 eq MgCl₂ | 1. CH₃CN, 20-80° C.; | 84% | 94:6 |
| | rac, 1 | 1.5 eq | 2. toluene/EtOAc/ | (HPLC) | |
| | eq | Mg(OPr-i)₂ | DBF, −20-20° C. | | |
| Example 5 | i- | 3.075 eq Mg, | 1. THF, 22-40° C.; | 79% | 94:6 |
| | Propyl, | 3.075 eq i- | 2. THF/toluene/DBF, | (HPLC) | |
| | rac, 1 | PrOH, | 20° C. | | |
| | eq | 0.5 mol % i- | | | |
| | | PrMgCl, 3.126 | | | |
| | | eq i-PrCl | | | |
| Example 6 | i-Butyl, | 3.0 eq | 1. THF, 25-35° C.; | 84% | 94:6 |
| | rac, 1 | MeMgCl | 2. THF/toluene/DBF, | (HPLC) | |
| | eq | (22% in THF) | 20° C. | | |
| | | 3.075 eq i- | | | |
| | | PrOH | | | |
| Example 7 | i-Butyl, | 3.0 eq | 1. THF, 25-35° C.; | 83% | 93:7 |
| | rac, 1 | MeMgCl | 2. THF/toluene/DBF, | (HPLC) | |
| | eq | (22% in THF) | 20° C. | | |
| | | 3.075 eq i- | | | |
| | | BuOH | | | |
| Example 8 | i-Butyl, | 3.075 eq Mg, | 1. THF, 22-40° C.; | 83% | 95:5 |
| | rac, 1 | 3.075 eq i- | 2. THF/toluene/DBF, | (HPLC) | |
| | eq | PrOH, | 20° C. | | |
| | | 0.5 mol % i- | | | |
| | | PrMgCl, 3.126 | | | |
| | | eq i-PrCl | | | |
| Example 9 | i-Butyl, | 3.075 eq Mg, | 1. THF, 22-40° C.; | 84% | 93:7 |
| | rac, 1 | 3.075 eq i- | 2. THF/toluene/DBF, | (HPLC) | |
| | eq | BuOH, | 20° C. | | |
| | | 0.5 mol % i- | | | |
| | | PrMgCl, 3.126 | | | |
| | | eq i-PrCl | | | |
| Example 10 | Methyl, | 3.0 eq | 1. THF, −20° C. to R.T. | 75% | 91:9 |
| | rac, 1 | MeMgCl | 2. THF/toluene, 15- | (HPLC) | |
| | eq | (22% in THF), | 20° C. | | |
| | | 3.075 eq | | | |
| | | CH₃CHO | | | |

Conditions from the prior art applied to the inventive reaction (Example 10)

| | | | | | |
|---|---|---|---|---|---|
| Carreira | Cyclic | 3.3 eq i-PrOH, | CH₂Cl₂, 0° C.-RT | 70% | 86:14 |
| ACIE 2001, | and | 3 eq EtMgBr | | | |
| 40, 2082, | acyclic | | | | |
| JACS 2001, | allyl | | | | |
| 123, 3611, | alcohols | | | | |
| OL 2007, 9, | | | | | |
| 3857 | | | | | |

Prior art (X₂₋₆ = H)

| | | | | | |
|---|---|---|---|---|---|
| Bull. Chem. | Methyl, | 2 eq EtMgBr | CH₂Cl₂, −78 to −30° C./ | 100 | 92:8/ |
| Soc. Jpn. | 2 eq | | to R.T. | | 89:11 |
| 1993, 2685- | Methyl, | 1 eq NEt₃ | CH₂Cl₂, −30° C. | 90 | 30:70 |
| 2689 | 1 eq | | | | |
| | Methyl, | 1 eq EtMgBr | THF/CH₂Cl₂, −78 to −30° | 93/86 | 63:37/ |
| | 1 eq | | C. | | 81:19 |

1. A process for preparing isoxazolinecarboxylic acid derivatives of formula (I) 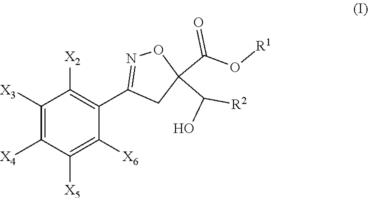
in which
X² is H, C₁-C₄ alkyl, C₁-C₄ fluoroalkyl, C₁-C₄ fluoroalkoxy, C₁-C₄ alkoxy, fluorine, or CN,
X³ is H, C₁-C₄ alkyl, C₁-C₄ fluoroalkyl, C₁-C₄ fluoroalkoxy, C₁-C₄ alkoxy, fluorine, chlorine, or CN,
X⁴ is H, C₁-C₄ alkyl, C₁-C₄ fluoroalkyl, C₁-C₄ fluoroalkoxy, C₁-C₄ alkoxy, fluorine, or CN,
X⁵ is H, C₁-C₄ alkyl, C₁-C₄ fluoroalkyl, C₁-C₄ fluoroalkoxy, C₁-C₄ alkoxy, fluorine, chlorine, or CN,
X⁶ is H, C₁-C₄ alkyl, C₁-C₄ fluoroalkyl, C₁-C₄ fluoroalkoxy, C₁-C₄ alkoxy, fluorine, or CN,
R¹ is H, or C₁-C₄-alkyl,
R² is C₁-C₄-alkyl,
characterized in that at least one compound of formula (II), (IIa), and (IIb) 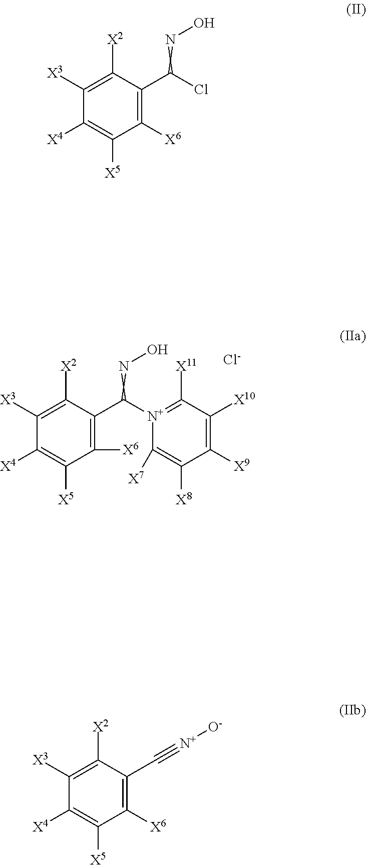
in which
X² to X⁶ have the definitions given above,
X⁷, X⁸, X¹⁰, X¹¹ are independently H or C₁-C₄-alkyl,
X⁹ is H, C₁-C₄-alkyl or N(C₁-C₄-alkyl)₂,
undergo a reaction with compounds of formula (III) 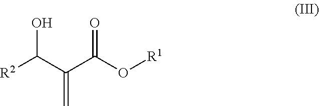
in which
R¹ and R² have the definitions given above,
with addition of a combination of reagents that enables formation of 2.0 to 4.5 equivalents of a reactive species “R³OMgHal” (IV)-based on compounds of the general formula (II), (IIa), and (IIb), where
R³ is C₂-C₆-alkyl, unsubstituted or C₁-C₆-alkyl-substituted benzyl and
Hal is a halogen,
to give compounds of the general formula (I).

2. The process according to claim 1, wherein the definitions of the radicals in the compounds of the general formulae (I), (II), (IIa), (IIb), and (III) are as follows:
X² is H, methyl, trifluoromethyl, difluoromethyl, difluoromethoxy, trifluoromethoxy, fluorine, methoxy, or CN,
X³ is H, methyl, trifluoromethyl, difluoromethyl, difluoromethoxy, trifluoromethoxy, fluorine, chlorine, methoxy, or CN,
X⁴ is H, methyl, trifluoromethyl, difluoromethyl, difluoromethoxy, trifluoromethoxy, fluorine, methoxy, or CN,
X⁵ is H, methyl, trifluoromethyl, difluoromethyl, difluoromethoxy, trifluoromethoxy, fluorine, chlorine, methoxy, or CN,
X⁶ is H, methyl, trifluoromethyl, difluoromethyl, difluoromethoxy, trifluoromethoxy, fluorine, methoxy, or CN,
R¹ is H, methyl, ethyl, i-propyl, or i-butyl,
R² is methyl or ethyl.

3. The process according to claim 2, wherein the definitions of the radicals in the compounds of the general formulae (I), (II), (IIa), (IIb), and (III) are as follows:
X² is H,
X³ is H, methyl, trifluoromethyl, difluoromethyl, fluorine, chlorine, methoxy, or CN,
X⁴ is fluorine or H,
X⁵ is H, methyl, trifluoromethyl, difluoromethyl, fluorine, chlorine, methoxy, or CN,
X⁶ is H,
R¹ is H, methyl, i-propyl, or i-butyl,
R² is methyl.

4. The process according to claim 3, wherein the definitions of the radicals in the compounds of the general formulae (I), (II), (IIa), (IIb), and (III) are as follows:
X² is H,
X³ is H or fluorine,
X⁴ is H or fluorine,
X⁵ is H or fluorine,
X⁶ is H,
R¹ is H, methyl, or i-butyl,
R² is methyl.

5. The process according to claim 4, wherein the definitions of the radicals in the compounds of the general formulae (I), (II), (IIa), (IIb), and (III) are as follows:
X² is H,
X³ is fluorine,
X⁴ is H,
X⁵ is fluorine,
X⁶ is H,
R¹ is H or methyl,
R² is methyl.

6. The process according to claim 1, wherein the definitions of the radicals in the compounds of the general formula (IIa) are as follows:
X⁷, X⁸, X¹⁰, X¹¹ are independently H, methyl, or ethyl,
X⁹ is H, methyl, ethyl, or N(methyl)₂.

7. The process according to claim 1, wherein the definitions of the radicals in the compounds of the general formula (IIa) are as follows:
X⁷, X¹¹ are H,
X⁸, X¹⁰ are independently H, methyl, or ethyl,
X⁹ is H, methyl, ethyl, or N(methyl)₂.

8. The process according to claim 1, wherein the definitions of the radicals in the compounds of the general formula (IIa) are as follows:
X⁸ is H, methyl, or ethyl,
X⁷, X¹¹ are independently H or methyl,
X⁹, X¹⁰ are H.

9. The process according to claim 1, wherein the compound of the general formula (IV) is generated by a combination of reagents, wherein the combination of reagents is one of:
R⁴MgHal and R³OH or
MgHal₂ and R³OM or
MgHal₂ and Mg(OR³)₂, where
R³ is C₂-C₆-alkyl, benzyl, or methylbenzyl,
Hal is a halogen,
M is an alkali metal,
R⁴ is C₁-C₆-alkyl, aryl, benzyl, allyl, or vinyl.

10. The process according to claim 1, wherein the compound of the general formula (IV) is generated by a combination of reagents, wherein the combination of reagents is one of:
R⁴MgHal and R³OH or
MgHal₂ and R³OM or
MgHal₂ and Mg(OR³)₂, where
R³ is C₂-C₄-alkyl,
Hal is bromine or chlorine,
M is an alkali metal,
R⁴ is C₁-C₄-alkyl, phenyl, benzyl, p-tolyl, or vinyl.

11. The process according to claim 1, wherein the compound of the general formula (IV) is generated by a combination of reagents, wherein the combination of reagents is one of:
R⁴MgHal and R³OH or
MgHal₂ and R³OM or
MgHal₂ and Mg(OR³)₂, where
R³ is i-propyl or i-butyl,
Hal is bromine or chlorine,
M is sodium,
R⁴ is methyl, ethyl, n-butyl, or i-propyl.

12. The process according to claim 1, characterized in that 2.8 to 3.2 equivalents of the reactive species “R³OMgHal” (IV) are used, based on compounds of the general formula (II), (IIa), and (IIb).

13. The process according to claim 1, characterized in that the solvent is toluene, xylene, tetrahydrofuran (THF), isopropyl acetate (i-PrOAc), acetonitrile, methyl tert-butyl ether (MTBE), methyl-THF, or ethyl acetate (EtOAc), or mixtures in any ratios thereof.

14. The process according to claim 1, characterized in that the reaction is conducted at −25° C. to 70° C.

15. The process according to claim 14, characterized in that the reaction is conducted at 10° C. to 30° C.

16. The process according to claim 1, characterized in that a diastereomeric ratio is increased by a further crystallization step.

17. The process according to claim 1, characterized in that the compounds of the general formula (IV) are generated via a Grignard reaction, specifically with the following combinations of reagents:
R⁵MgHal and R⁶R⁷CO, where
R⁵ is C₁-C₆-alkyl, aryl, or benzyl,
R⁶, R⁷ are each one of H, C₁-C₆-alkyl, aryl, and the resulting radical definition
R³ is R⁵R⁶R⁷C.

18. A mixture comprising a first compound of formula (Ia) and a second compound of formula (Ib), wherein the first and second compounds are present in unequal amounts, wherein the radicals have the definitions according to claim 3, excluding methyl 3-(3,5-difluorophenyl)-5-(1-hydroxyethyl)-4H-isoxazole-5-carboxylate and methyl 3-phenyl-5-(1-hydroxyethyl)-4H-isoxazole-5-carboxylate:

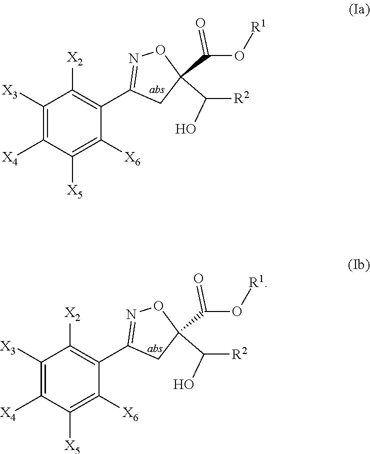

19. A mixture comprising a first compound of formula (Iaa) and a second compound of formula (Iba), wherein the first and second compounds are present in unequal amounts, wherein the radicals have the definitions according to claim 3, excluding methyl 3-(3,5-difluorophenyl)-5-(1-hydroxyethyl)-4H-isoxazole-5-carboxylate and methyl 3-phenyl-5-(1-hydroxyethyl)-4H-isoxazole-5-carboxylate:

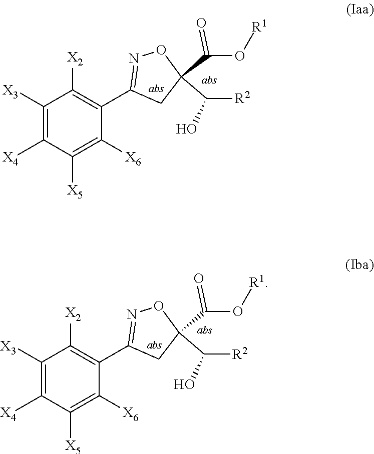

20. A mixture comprising a first compound of formula (Iab) and a second compound of formula (Ibb), wherein the first and second compounds are present in unequal amounts, wherein the radicals have the definitions according to claim 3, excluding methyl 3-(3,5-difluorophenyl)-5-(1-hydroxyethyl)-4H-isoxazole-5-carboxylate and methyl 3-phenyl-5-(1-hydroxyethyl)-4H-isoxazole-5-carboxylate: [chemical structure]
